(19) United States
(12) Patent Application Publication
(10) Pub. No.: US 2023/0127647 A1
(43) Pub. Date: Apr. 27, 2023

(54) PHOTODYNAMIC THERAPY TREATMENT SUPPORT DEVICE

(71) Applicants: NATIONAL CANCER CENTER, Tokyo (JP); Shimadzu Corporation, Kyoto-shi (JP)

(72) Inventors: Tomonori YANO, Kashiwa-shi (JP); Kenji TAKASHIMA, Kashiwa-shi (JP); Tomohiro MITSUI, Kashiwa-shi (JP); Maasa SASABE, Kashiwa-shi (JP); Akihiro ISHIKAWA, Kyoto-shi (JP)

(21) Appl. No.: 17/971,287

(22) Filed: Oct. 21, 2022

(30) Foreign Application Priority Data
Oct. 21, 2021 (JP) 2021-172559

(51) Int. Cl. A61N 5/06; A61K 41/00

(52) U.S. Cl. CPC A61N 5/062; A61K 41/0071; A61N 2005/0628; A61N 2005/0658

(57) ABSTRACT

A photodynamic therapy treatment support device is provided with a light source and a distribution information output unit. The light source irradiates a photosensitive substance administered to a body of a subject with light of a specific wavelength bandwidth having energy capable of generating fluorescence from the photosensitive substance without advancing necroses of tumor cells. The distribution information output unit outputs information on a distribution state of the fluorescence generated from the photosensitive substance, based on the fluorescence generated from the photosensitive substance.

### CROSS-REFERENCE TO RELATED APPLICATIONS

The related Japanese Patent Application No. 2021-172559, entitled “Photodynamic Therapy Treatment Support Device” filed on Oct. 21, 2021 and invented by Tomonori Yano, Kenji Takashima, Tomohiro Mitsui, Maasa Sasabe, and Akihiro Ishikawa, upon which this patent application is based is hereby incorporated by reference in its entirety.

### BACKGROUND OF THE INVENTION

### Field of the Invention

The present invention relates to a photodynamic therapy treatment support device.

### Description of the Background Art

Photodynamic therapy is conventionally known as a treatment method for treating malignant tumors, such as, e.g., esophageal cancer, lung cancer, and brain tumors. In photodynamic therapy, a photosensitive substance is initially administered as a medical agent that causes a photochemical reaction and accumulates in tumor cells to a patient. Then, in a state in which the photosensitive substance accumulates in the tumor cells and around the tumor cells, light of a specific wavelength bandwidth corresponding to the photosensitive substance is continuously emitted. This causes fluorescence emission from the photosensitive substance and a photochemical reaction to generate active oxygen (singlet). In the photodynamic therapy, the active oxygen generated based on the photochemical reaction of the photosensitive substance causes damage to the tumor cells in which the photosensitive substance is accumulated and also causes damage to the blood vessels around the tumor cells. This causes necroses of the tumor cells by interrupting the nutrition supply to the tumor cells by blood flow. Note that when irradiated with light of a specific wavelength bandwidth, the photosensitive substance that has caused the photochemical reaction (photochemical reaction has progressed) does not emit fluorescence.

Further, Japanese Unexamined Patent Application Publication No. 2014-221117 discloses a treatment progress monitor device. This treatment progress monitor device displays, in real time, an index that estimates the volume and the depth of the treatment region using the fluorescence intensity of the fluorescence generated from the photosensitive substance, during the treatment by the photodynamic therapy (while continuously emitting the light of the specific wavelength bandwidth according to the photosensitive substance).

Here, in the photodynamic therapy, in order to enhance the treatment effects, it is desirable to confirm the distribution state of the photosensitive substance (medical agent) prior to the treatment and initiate the treatment at a position in a state in which sufficient numbers of photosensitive substances are accumulated in the tumor cells of the patient. Further, in order to determine whether or not additional treatment is required after the treatment, there is a desire to confirm the presence or absence of the photosensitive substance that has not progressed the photochemical reaction and has not acted on the treatment from the distribution state of the fluorescence emitted from the photosensitive substance. Therefore, it is desired to be able to confirm the distribution state of the photosensitive substance accumulated in the tumor cells even in a state of not being treated (i.e., before the treatment or after the treatment). Note that the treatment by photodynamic therapy means that a photosensitive substance is administered to a subject, and then light of a specific wavelength bandwidth is continuously emitted to thereby provide energy for causing necroses of the tumor cancers to the photosensitive substance.

### SUMMARY OF THE INVENTION

The present invention has been made to solve the above-described problems. It is an object of the present invention to provide a treatment support device capable of confirming a distribution state of a photosensitive substance accumulated in tumor cells before treatment or after treatment by photodynamic therapy.

A photodynamic therapy treatment support device according to one aspect of the present invention for use in photodynamic therapy for causing necroses of tumor cells by active oxygen generated by a photochemical reaction caused by irradiating a photosensitive substance with light of a specific wavelength bandwidth, the photodynamic therapy treatment support device comprising:

a light source configured to irradiate the photosensitive substance administered to a body of a subject with the light of the specific wavelength band, the light having energy capable of generating fluorescence from the photosensitive substance without causing necroses of the tumor cells; and

a distribution information output unit configured to output information on a distribution state of the fluorescence generated from the photosensitive substance, based on the fluorescence generated from the photosensitive substance.

In the photodynamic therapy treatment support device according to one aspect of the present invention, the light source emits the light of the specific wavelength bandwidth having energy capable of generating fluorescence from the photosensitive substance administered to the body of the subject without causing necroses of tumor cells. The distribution information output unit outputs information on the distribution state of the fluorescence generated from the photosensitive substance, based on the fluorescence generated from the photosensitive substance. As a result, information on the distribution state of the fluorescence generated from the photosensitive substance is outputted without causing necroses of the tumor cells (without advancing the treatment) before the treatment or after the treatment. Therefore, it is possible to confirm the distribution state of the photosensitive substance accumulated in the tumor cells before the treatment or after the treatment by photodynamic therapy.

### BRIEF DESCRIPTION OF THE DRAWINGS

FIG. 1 is a diagram for explaining photodynamic therapy.

FIG. 2 is a diagram showing a spectrum of fluorescence of Laserphyrin (registered trademark).

FIG. 3 is a first diagram for explaining the mechanism of the photosensitive substance in photodynamic therapy.

FIG. 4 is a second diagram for explaining the mechanism of the photosensitive substance in photodynamic therapy.

FIG. 5 is a diagram showing an example of the change in the fluorescence intensity during the treatment.

FIG. 6 is a block diagram illustrating an entire configuration of a treatment support device according to one embodiment of the present invention.

FIG. 7 is a diagram showing one example of a fluorescence distribution image.

FIG. 8 is a diagram showing one example of a visible light image.

FIG. 9 is a diagram showing one example of a composite image.

FIG. 10 is a diagram showing one example of the decreasing tendency in the fluorescence intensity due to the increase in the integrated energy amount.

FIG. 11 is a diagram showing one example of the irradiation intensity and the irradiation time of the excitation light emitted when generating a fluorescence distribution image before the treatment or after treatment by a treatment support device according to one embodiment of the present invention.

FIG. 12 is a diagram showing another example of the irradiation intensity and the irradiation time of the excitation light emitted when generating a fluorescence distribution image before the treatment or after the treatment.

FIG. 13 is a block diagram showing an entire configuration of a treatment support device according to a first modification.

FIG. 14 is a block diagram showing an entire configuration of a treatment support device according to a second modification.

### DESCRIPTION OF THE PREFERRED EMBODIMENTS

Hereinafter, some embodiments in which the present invention is embodied will be described with reference to the attached drawings.

### (Photodynamic Therapy)

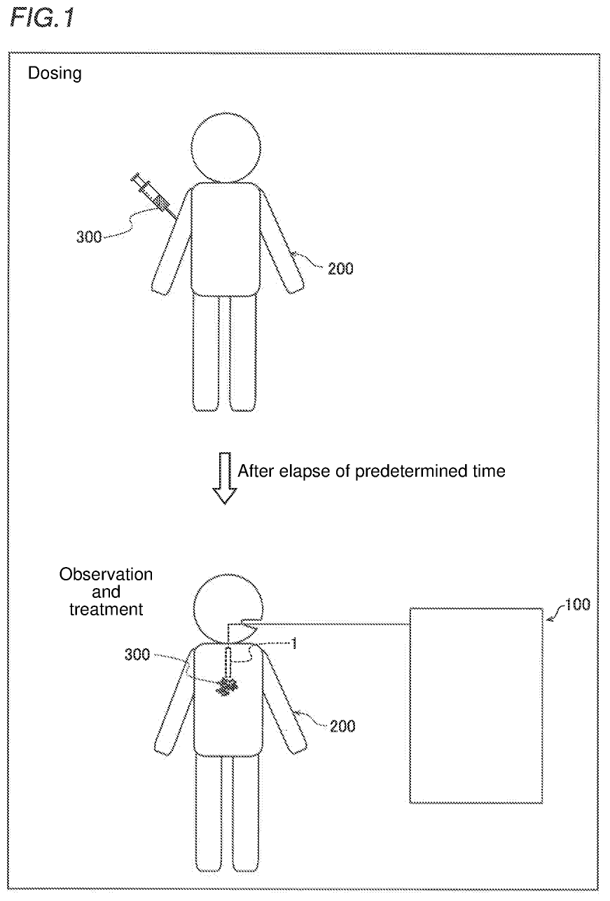

First, with reference to FIG. 1 to FIG. 5, photodynamic therapy (PDT: Photodynamic Therapy) in which a treatment support device 100 according to one embodiment of the present invention is used will be described. In photodynamic therapy, as shown in FIG. 1, a medical agent that is a photosensitive substance 300 is administered to a body of a patient 200 prior to treatment. After administering the photosensitive substance 300 (medical agent) to the patient 200, the observation and the treatment are performed using the treatment support device 100, after the lapse of a predetermined period of time (4-6 hours) during which the photosensitive substance 300 (medical agent) accumulates in the tumor cells 201. Note that the patient 200 is one example of the “subject” recited in claims.

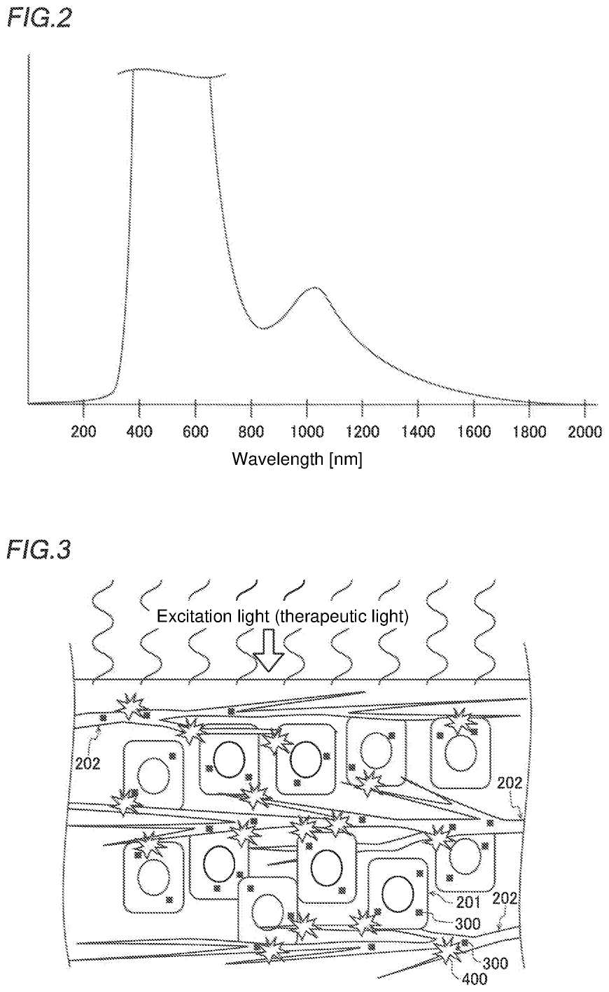

The photosensitive substance 300 is a material that emits fluorescence by being excited by irradiation of light of a specific wavelength bandwidth. The photosensitive substance 300 used in this embodiment is talaporfin sodium. Specifically, Laserphyrin (registered trademark) is used as a medical agent that is the photosensitive substance 300. Laserphyrin (registered trademark) is excited by light having a wavelength of 664 nm to emit light having a wavelength of 672 nm to 1,800 nm as fluorescence as shown in FIG. 2. That is, in the case of using Laserphyrin (registered trademark) as a medical agent, the light of the specific wavelength bandwidth is light of a wavelength bandwidth including 664 nm.

The photosensitive substance 300 administered to the patient 200 passes through the blood vessel 202 to circulate the body of the patient 200 by the blood flow and is absorbed by the cells in the body to be accumulated therein. The photosensitive substance 300 is discharged out of the cells over time. However, in the tumor cells 201, the time from the absorption of the photosensitive substance 300 to the discharge thereof is longer as compared with normal cells. For this reason, it is possible to attain a state in which the photosensitive substance 300 has been selectively accumulated in the tumor cells 201 and therearound by using the time lag from the absorption of the photosensitive substance 300 to the discharge thereof between tumor cells 201 and normal cells.

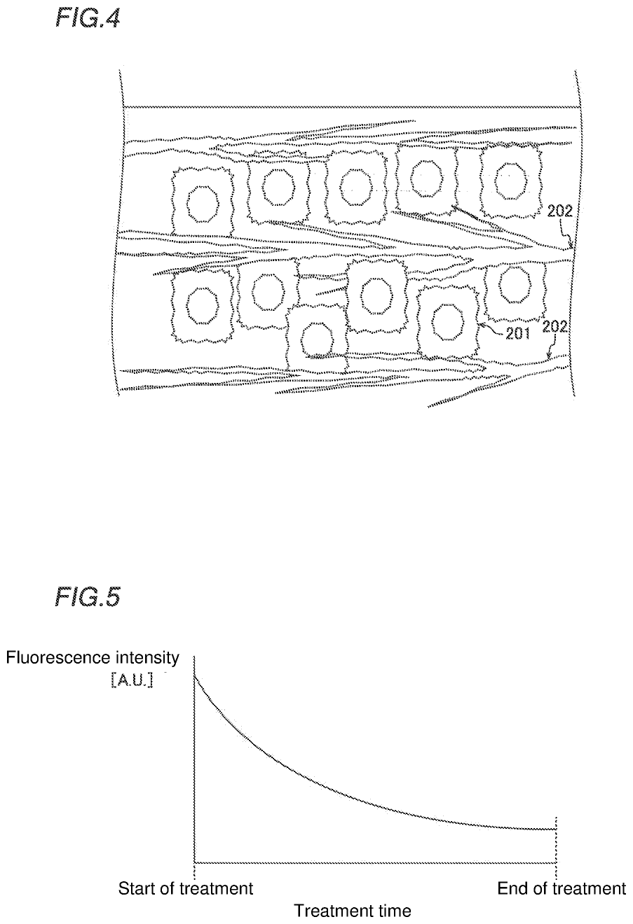

Further, the photosensitive substance 300 is a material that causes photochemical reactions by being continuously irradiated with light of a specific wavelength bandwidth (the light of the specific wavelength bandwidth is integrated). When the photosensitive substance 300 changes from the excited state to the ground state, the excited energy is applied to the oxygen in the tumor cells 201 and therearound, thereby generating active oxygen 400 (singlet oxygen) (see FIG. 3). The active oxygen 400 generated based on the photochemical reaction of the photosensitive substance 300 damages the tumor cells 201 in which the photosensitive substance 300 is accumulated, by the oxidative power, and also damages blood vessels 202 around the tumor cells 201. With this, the supply of nutrients to the tumor cells 201 by the blood flow is interrupted. Consequently, the tumor cells 201 necrotize (see FIG. 4). In the treatment by the photodynamic therapy, in a state in which the photosensitive substance 300 has been selectively accumulated in the tumor cells 201 and therearound, light of a specific wavelength bandwidth according to the photosensitive substance 300 is kept irradiated. With this, the photosensitive substance 300 is made to cause a photochemical reaction to selectively necrotize the tumor cells 201 by the active oxygen 400 generated based on the photochemical reaction of the photosensitive substance 300. Note that the photosensitive substance 300 does not emit fluorescence after being changed by the photochemical reaction (after the photochemical reaction has progressed). Thus, as shown in FIG. 5, as the treatment progresses, the fluorescence intensity to be detected decreases.

### (Configuration of Treatment Support Device)

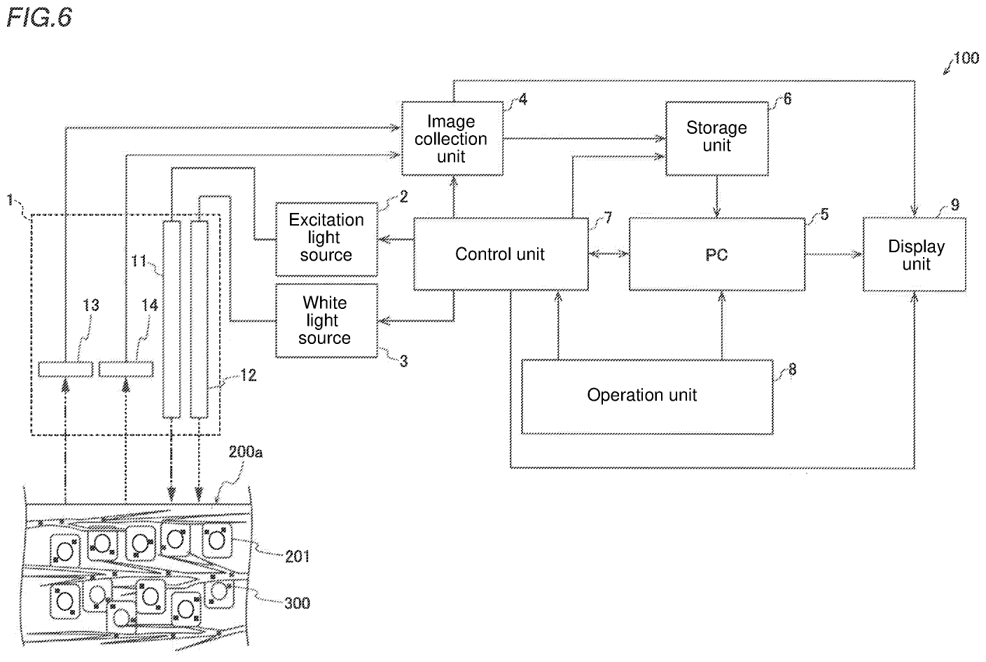

With reference to FIG. 6, the configuration of the treatment support device 100 according to this embodiment will be described.

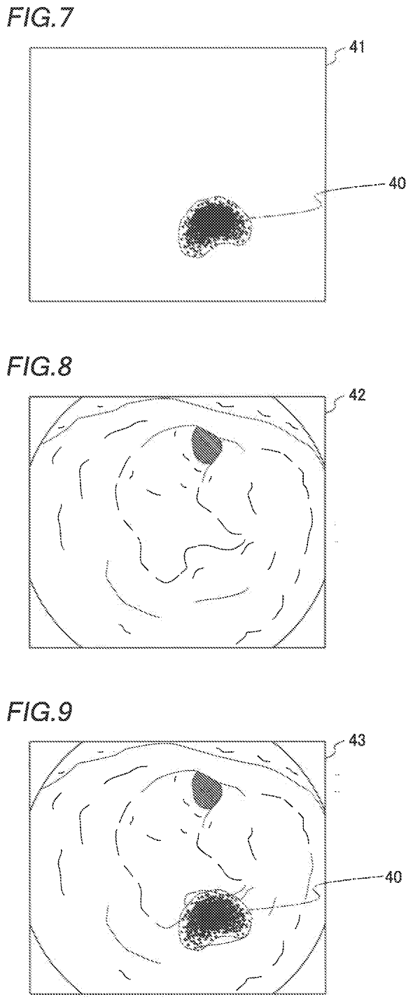

The treatment support device 100 according to this embodiment is a device for supporting treatment in photodynamic therapy as described above. Note that the treatment support device 100 is one example of the “photodynamic therapy treatment support device” recited in claims. The treatment support device 100 according to this embodiment emits light (excitation light) of a specific wavelength bandwidth for exciting the photosensitive substance 300 to an affected area 200a of the patient 200 and detects the fluorescence emitted from the photosensitive substance 300 administered to the patient 200. As will be described later, the treatment support device 100 is configured to generate the fluorescence distribution image 41 (see FIG. 7), which is an image representing the distribution state of the fluorescence, by the detected fluorescence.

By using the treatment support device 100 according to this embodiment, the user can confirm, prior to the treatment by the photodynamic therapy, how and to what extent the photosensitive substance 300 has been distributed and accumulated in the affected area 200a (see FIG. 6) in the body of the patient 200. The phrase “prior to the treatment by the photodynamic therapy” means prior to applying energy for causing necroses of tumor cells 201 to the photosensitive substance 300 by continuously emitting light of a specific wavelength bandwidth. Further, the user can grasp, by the treatment support device 100, the effects of the treatment from the distribution and the accumulation of the photosensitive substance 300 in the affected area 200a, after the treatment by the photodynamic therapy.

Further, the treatment support device 100 is configured to be able to perform the treatment by the photodynamic therapy, in addition to the support of the treatment by the photodynamic therapy, such as, e.g., a display of the distribution state of the fluorescence. Here, the above-described “treatment by the photodynamic therapy” refers to necrotizing of the tumor cells 201 by continuously emitting light of a specific wavelength bandwidth according to the photosensitive substance 300.

As shown in FIG. 6, the treatment support device 100 is provided with an endoscope 1, an excitation light source 2, a white light source 3, an image collection unit 4, a Personal Computer (PC) 5, and a storage unit 6. Note that the excitation light source 2 is one example of the “light source” as recited in claims. Further, the image collection unit 4 is one example of the “distribution information output unit” as recited in claims. The PC 5 is one example of the “image composition unit” as recited in claims. Further, the treatment support device 100 is further provided with a control unit 7, an operation unit 8, and a display unit 9.

Further, the endoscope 1 of the treatment support device 100 is provided with light guides 11 and 12, a fluorescence detection unit 13, and a visible light detection unit 14. The endoscope 1 is provided with an objective lens, an air blower, a treatment tool, and the like (not shown). Note that the endoscope 1 is configured to be inserted into the body of the patient 200 by a doctor (user).

The excitation light source 2 is configured to emit light of a specific wavelength bandwidth capable of generating fluorescence from the photosensitive substance 300. That is, the excitation light source 2 is configured to emit the light of the specific wavelength bandwidth for exciting the photosensitive substance 300. The excitation light source 2 includes a Laser Diode (LD), a Light Emitting Diode (LED), or the like. The specific wavelength bandwidth of the light (therapeutic light) emitted from the excitation light source 2 when exciting the photosensitive substance 300 before the treatment or after the treatment may be the same wavelength bandwidth as the specific wavelength bandwidth of the light (excitation light) emitted to the photosensitive substance 300 at the time of the treatment, or may be a wavelength bandwidth not partially overlapped. Further, the half width at half maximum of the spectrum of the excitation light may be different from the half width at half maximum of the spectrum of the therapeutic light.

The light guide 11 of the endoscope 1 is configured to guide the excitation light from the excitation light source 2 to emit it. The light guide 11 includes an optical fiber. The light of the specific wavelength bandwidth emitted from the excitation light source 2 is emitted to the affected area 200a via the light guide 11 to excite the photosensitive substance 300. Note that the light guide 11 may include a laser catheter.

The treatment support device 100 can also perform treatment by photodynamic therapy by continuously emitting the light of the specific wavelength bandwidth emitted from the excitation light source 2 to the affected area 200a (photosensitive substance 300) of the patient 200 from the endoscope 1, as previously described.

Note that the light emitted to the photosensitive substance 300 by the endoscope 1 is light of a wavelength bandwidth that excites the photosensitive substance 300 (medical agent) used for the treatment to cause a photochemical reaction. This light varies depending on the type of the photosensitive substance 300 (medical agent) used for the treatment. For example, in the case of using Laserphyrin (registered trademark) described above as the photosensitive substance 300 (medical agent), the light emitted at the time of the treatment, before the treatment, and after the treatment is light of a wavelength bandwidth including a wavelength of 664 nm.

The white light source 3 is a light source that emits visible light and is configured to emit white light. The white light source 3 includes a light-emitting diode or the like. The light guide 12 of the endoscope 1 is configured to guide the white light (visible light) from the white light source 3. The light guide 12 includes an optical fiber. The white light emitted from the white light source 3 is emitted to the affected area 200a of the patient 200 via the light guide 12 of the endoscope 1 in order to illuminate the affected area 200a of the patient 200 to detect the visible light (reflected light) reflected from the affected area 200a of the patient 200. Note that the light guide 12 may include a laser catheter.

The fluorescence detection unit 13 is configured to detect the fluorescence emitted by the photosensitive substance 300 by the irradiation of the light of a specific wavelength bandwidth. The fluorescence detection unit 13 is an image sensor for detecting the fluorescence emitted from the photosensitive substance 300. The fluorescence detection unit 13 images the fluorescence emitted from the photosensitive substance 300 at a predetermined frame rate, such as, e.g., a frame rate of an NTSC (National Television System Committee) standard. The fluorescence detection unit 13 includes an image sensor, such as, e.g., a CMOS (Complementary metal Oxide Semiconductor) image sensor or a CCD (Charge Coupled Device) image sensor.

Further, the fluorescence detection unit 13 is configured to detect the light having a wavelength longer than the wavelength of the light of the specific wavelength bandwidth emitted by the excitation light source 2, without detecting the light of the specific wavelength bandwidth emitted from the excitation light source 2 and light having a wavelength shorter than the wavelength of the specific wavelength bandwidth. Specifically, the fluorescence detection unit 13 is configured to selectively detect light having a wavelength longer than the wavelength of the light of the specific wavelength bandwidth emitted from the excitation light source 2 by the wavelength selectivity of the optical filter. For example, in the case where Laserphyrin (registered trademark) is used as the photosensitive substance 300, it is configured such that light having a wavelength longer than a wavelength (672 nm) of light excited by Laserphyrin (registered trademark) is selectively detected by the wavelength selectivity of the optical filter.

In this embodiment, the fluorescence detection unit 13 is configured to detect the light of the specific wavelength bandwidth of the fluorescence emitted from the photosensitive substance 300, without detecting the light of the specific wavelength bandwidth emitted from the excitation light source 2 and light having a wavelength shorter than the wavelength of the light of the specific wavelength bandwidth. Specifically, the fluorescence detection unit 13 is configured to selectively detect the light in a region including the wavelength bandwidth of the fluorescence emitted by the photosensitive substance 300 by the wavelength selectivity of the optical filter. For example, in a case where Laserphyrin (registered trademark) is used as the photosensitive substance 300, the fluorescence detection unit 13 is configured to detect the fluorescence based on the light of a wavelength bandwidth of 672 nm or more and 1,800 nm or less by the wavelength selectivity of the optical filter. In the case of exciting Laserphyrin (registered trademark), the light of a wavelength bandwidth including a wavelength of 664 nm is emitted. Therefore, the fluorescence detection unit 13 is preferably configured to detect the fluorescence, based on the light of the wavelength bandwidth of 750 nm or more and 1,800 nm or less excluding the vicinity of 664 nm, by the wavelength selectivity of the optical filter.

The visible light detection unit 14 is configured to detect the visible light emitted from the light guides 11 and 12 of the endoscope 1 and reflected from the affected area 200a of the patient 200. The visible light detection unit 14 is an image sensor for detecting the visible light (reflected light) reflected from the affected area 200a of the patient 200. The visible light detection unit 14 includes an image sensor, such as, e.g., a CMOS image sensor and a CCD-image sensor. The visible light detection unit 14 images the visible light (reflected light) reflected from the affected area 200a of the patient 200 at a predetermined frame rate, such as, e.g., a frame rate of an NTSC standard. Further, in the case of using Laserphyrin (registered trademark) as the photosensitive substance 300, the visible light detection unit 14 detects the visible light including light (excitation light and therapeutic light) of the specific wavelength bandwidth emitted from the excitation light source 2.

The image collection unit 4 includes a processor, such as, e.g., a GPU (Graphics Processing Unit) or an FPGA (Field-Programmable Gate Array) configured for image processing.

The image collection unit 4 receives the signal outputted from the fluorescence detection unit 13 based on the detected fluorescence. That is, it is configured such that the fluorescence signal detected by the fluorescence detection unit 13 is inputted to the image collection unit 4 as an electric signal. Further, the image collection unit 4 receives a signal outputted from the visible light detection unit 14 based on the detected visible light. That is, it is configured such that the signal of the visible light detected by the visible light detection unit 14 is inputted to the image collection unit 4 as an electric signal. The image collection unit 4 is configured to collect the fluorescence signal and the signal of the visible light based on the time series. The image collection unit 4 is configured to collect or stop collecting the fluorescence signal and collect or stop collecting the visible light under the control of the control unit 7.

The image collection unit 4 is configured to output the information on the distribution state of the fluorescence emitted from the photosensitive substance 300, based on the fluorescence generated from the photosensitive substance 300. In this embodiment, the image collection unit 4 is configured to generate a fluorescence distribution image 41 (see FIG. 7), which is an image representing the distribution state of the fluorescence generated from the photosensitive substance 300, based on the fluorescence generated from the photosensitive substance 300. That is, in this embodiment, the image collection unit 4 is configured to output the fluorescence distribution image 41 as the information on the distribution state of the fluorescence generated from the photosensitive substance 300.

Further, in this embodiment, the image collection unit 4 is configured to generate the visible light image 42 (see FIG. 8) based on the visible light detected by the visible light detection unit 14.

The PC 5 is a computer including a CPU (Central Processing Unit), a ROM (Read Only Memory), a RAM (Random Access Memory), etc.

The PC 5 is configured to generate a composite image 43 (see FIG. 9) by superimposing the fluorescence distribution image 41 (image data of the fluorescence distribution image 41) generated by the image collection unit 4 and the visible light image 42 (image data of the fluorescence distribution image 41) generated by the image collection unit 4. That is, the PC 5 is configured to generate the composite image 43 in which the plurality of images generated by the image collection unit 4 is superimposed. Note that the PC 5 may include the image collection unit 4 as a functional configuration (functional block). That is, the PC 5 may be configured to function as the image collection unit 4 by executing a program.

The PC 5 is configured to analyze the fluorescence signal and the signal of the visible light (data of the fluorescence distribution image 41 and the visible light image 42) collected by the image collection unit 4. That is, the PC 5 is configured to analyze the fluorescence signal (fluorescence signal value) detected by the fluorescence detection unit 13. The PC 5 is configured to analyze the fluorescence signal value on a time series basis. Further, the PC 5 is configured to analyze the signal value of the visible light detected by the visible light detection unit 14.

The storage unit 6 is configured to be capable of storing the image data, such as, e.g., the fluorescence distribution image 41, the visible light image 42, and the composite image 43. The storage unit 6 includes a storage device, such as, e.g., a non-volatile memory, a hard disk drive (HDD: Hard Disk Drive), and an SSD (Solid State Drive). Note that the storage unit 6 may include a database on a network connected by the network provided outside the treatment support device 100. The storage unit 6 is configured to memorize (store) the data of the fluorescence signal and the visible light collected by the image collection unit 4. The storage unit 6 memorizes (stores) the signal (the data of the fluorescence distribution image 41 and the visible light image 42) including the fluorescence signal and the therapeutic light collected on the time series basis by the image collection unit 4, together with a time stamp, such as, e.g., an imaging date and time. The storage unit 6 is configured to store programs to be executed by the control unit 7 to control the irradiation of light (excitation light and therapeutic light) of a specific wavelength bandwidth and data required to control the irradiation of light (excitation light and therapeutic light) of a specific wavelength bandwidth, when generating the fluorescence distribution image 41.

The control unit 7 includes a control board (circuit board) on which a CPU, a ROM, a RAM, and the like are mounted. The control unit 7 is configured to control the entire treatment support device 100. The control unit 7 is communicably connected to each of the excitation light source 2, the white light source 3, the image collection unit 4, the PC 5, the storage unit 6, the operation unit 8, and the display unit 9. Note that the PC 5 and the control unit 7 may be integrally formed.

The control unit 7 is configured to perform control of the irradiation of the light (excitation light and therapeutic light) of a specific wavelength bandwidth by the excitation light source 2. Specifically, the control unit 7 is configured to control the excitation light source 2 to turn on and off the excitation light source 2 (start emitting and stop emitting the light of the specific wavelength bandwidth). Further, the control unit 7 is configured to control the excitation light source 2 to control the irradiation time and the irradiation intensity of the light (excitation light and therapeutic light) of the specific wavelength bandwidth.

Further, the control unit 7 is configured such that a doctor (user) can perform control, such as, e.g., starting the irradiation of the light of the specific wavelength bandwidth and stopping the irradiation of the light of the specific wavelength bandwidth (switching ON/OFF of the irradiation) by manipulating the operation unit 8. Similarly to the control of the excitation light source 2, the control unit 7 is configured to control to turn on and off the white light source 3 and control the irradiation time and the irradiation intensity of the white light.

The control unit 7 is configured to perform control to display the image, such as, e.g., the fluorescence distribution image 41, the visible light image 42, and the composite image 43 on the display unit 9.

The operation unit 8 is a user interface for operating the treatment support device 100. The operation unit 8 includes, for example, a remote control, a touch panel, a keyboard, a mouse, or the like. Further, a touch panel as the operation unit 8 may be provided on the display unit 9. That is, the operation unit 8 and the display unit 9 may be integrally formed. Further, the operation unit 8 may be separately provided corresponding to each of the PC 5 and the control unit 7.

The operation unit 8 is configured to accept operations related to the control of the treatment support device 100. Specifically, the operation unit 8 is configured to accept an operation for turning on and off the excitation light source 2 (switching on and off the irradiation of the light of the specific wavelength bandwidth), and an operation for turning on and off the white light source 3 (switching on and off the irradiation of the white light). Further, the operation unit 8 is configured to accept the operation for starting and stopping the detection of the fluorescence (acquisition of the fluorescence signal), the operation for starting and stopping the detection of the visible light (acquisition of the visible light), and the operation for switching the display method of the image to be displayed on the display unit 9.

Further, the operation unit 8 is configured to accept an operation to the PC 5 that analyzes the fluorescence signal and the visible light signal (data of the fluorescence distribution image 41 and the visible light image 42). Further, the operation unit 8 is configured to accept an operation for setting the region of interest (ROI: Region Of Interest) for selectively acquiring the fluorescence signal.

The display unit 9 is configured by, for example, a liquid crystal display or an organic EL (electroluminescence) display. The display unit 9 is connected to the PC 5 and the control unit 7 by, for example, a video interface, such as, e.g., an HDMI.

The composite image 43 is displayed on the display unit 9 under the control of the control unit 7. Further, the display unit 9 is configured to be capable of displaying the fluorescence distribution image 41 and the visible light image 42 in addition to the composite image 43, under the control of the control unit 7. Specifically, the treatment support device 100 can display each of the fluorescence distribution image 41, the visible light image 42, and the composite image 43 on the display unit 9 by switching them. The treatment support device 100 can display any one or two or all of the fluorescence distribution image 41, the visible light image 42, and the composite image 43 on the display unit 9 at the same time.

In the treatment support device 100 according to this embodiment, the user can observe, prior to the treatment, the distribution 40 (see FIG. 7) of the fluorescence in the fluorescence distribution image 41 or the distribution 40 (see FIG. 9) of the fluorescence in the composite image 43 displayed on the display unit 9. Therefore, in the body of the patient 200 (affected area 200a), it is possible to confirm how the photosensitive substance 300 is distributed and how much it is accumulated.

Further, in the treatment support device 100, for the purpose of making it easier for the user to confirm the distribution 40 of the fluorescence in the composite image 43, in the composite image 43, the superimposed visible light image 42 and distribution 40 of the fluorescence are displayed in an identifiable manner. Specifically, the control unit 7 sets the display color of the fluorescence distribution 40 to a color that is easily distinguishable from the visible light image 42. The display color of the fluorescence distribution 40 is set to, for example, fluorescence colors, such as, e.g., green, purple, or blue, or a color other than a color of the body surface, the tissues, and the blood of the patient 200. Alternatively, the control unit 7 makes it easier to distinguish between the visible light image 42 and the fluorescence distribution 40 by blinking the distribution 40 of the fluorescence. In this manner, the treatment support device 100 generates the composite image 43 in which the visible light image 42 and the distribution 40 of the fluorescence are distinguishable by the PC 5 and displays it on the display unit 9.

Further, it may be configured such that the PC 5 sets a threshold to the detected fluorescence intensity, the total value of the detected fluorescence intensity, or the like, in advance, and performs control to change the display method of the fluorescence distribution 40 or control to display notifying that it has exceeded the threshold, in a case where the detected fluorescence intensity or the total value of the detected fluorescence intensity or the like become equal to or larger than the threshold. With this, the user can easily grasp the portion (affected area 200a) in which the photosensitive substance 300 has been accumulated.

Then, in the treatment support device 100 according to this embodiment, it is possible to start the treatment for necrotizing tumor cells 201 by continuously irradiating the affected area 200a in which the photosensitive substance 300 has been accumulated with the light of the specific wavelength bandwidth, from the endoscope 1 inserted into the body of the patient 200. Note that the photosensitive substance 300 will not emit fluorescence after being changed by the photochemical reaction, and therefore, the fluorescence distribution 40 varies as the treatment proceeds. That is, from the change in the fluorescence signal during the treatment, the change in the distribution 40 of the fluorescence in the fluorescence distribution image 41, and the change in the distribution 40 of the fluorescence in the composite image 43, the user can grasp the distribution of the photosensitive substance 300 in the body of the patient 200 and the progress of the treatment. Further, the user can grasp the effects of the treatment (whether or not the treatment has progressed as expected) by confirming, similar to before the treatment, the change in the fluorescence signal during the treatment, the change in the distribution 40 of the fluorescence in the fluorescence distribution image 41, and the change in the distribution 40 of the fluorescence in the composite image 43.

Note that in this embodiment, the light (therapeutic light) of the specific wavelength bandwidth to be emitted for treatment is configured to be emitted from the excitation light source 2, also serving as excitation light. Note that the light (therapeutic light) of the specific wavelength bandwidth to be emitted for the treatment may be emitted from a light source different from the excitation light source 2 or may be emitted from a device different from the treatment support device 100.

### (Configuration of Irradiation of Excitation Light)

In photodynamic therapy, the excitation light source 2 is configured to irradiate the photosensitive substance 300 administered to the body of the patient 200 with the light of the specific wavelength bandwidth having energy capable of generating fluorescence from the photosensitive substance 300 without causing necroses of tumor cells 201 (progressing the treatment). In this embodiment, prior to the treatment or after the treatment by the photodynamic therapy, the excitation light source 2 emits the light of the specific wavelength bandwidth having energy capable of generating fluorescence from the photosensitive substance 300 without causing necroses of the tumor cells 201 (progressing the treatment), to the photosensitive substance 300 administered to the body of the patient 200.

The energy that does not cause necroses of the tumor cells 201 can be confirmed in advance by testing or the like. For example, it is possible to confirm the integrated amount of the energy that does not cause necroses of the tumor cells 201 (does not cause the start of necroses of the tumor cells 201), by observing the state of the tumor cells 201 with microscopy or the like while emitting the light of a wavelength bandwidth that causes a photochemical reaction due to excitation of the photosensitive substance 300 to the tumor cells 201 in which the photosensitive substance has been accumulated. For example, in a case where the integrated amount of the energy to be given at the time of the treatment is about 100 J/cm² and the integrated amount of the energy that does not cause necroses of the tumor cells 201 is about 1/100, the integrated amount of energy that does not cause necroses of the tumor cells 201 is about 1 J/cm². Note that the integrated amount of energy can be obtained from the irradiation intensity of the light (excitation light) of the specific wavelength bandwidth emitted from the excitation light source 2 and the irradiation time of the light (excitation light) of the specific wavelength bandwidth emitted from the excitation light source 2.

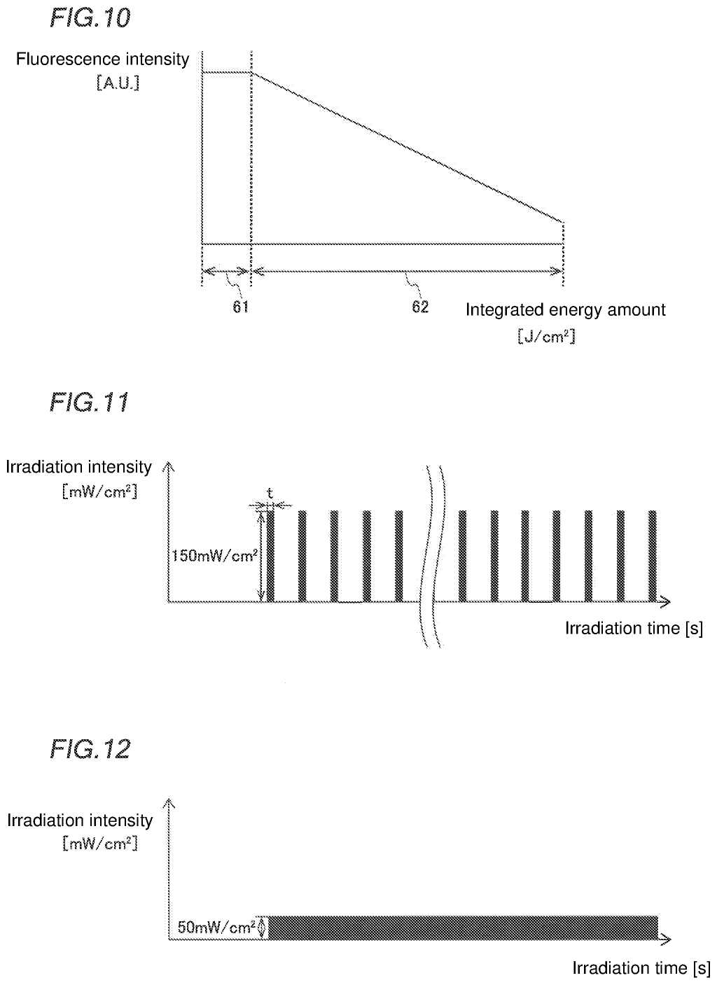

The control unit 7 is configured to control the irradiation of the light (excitation light) of the specific wavelength bandwidth by the excitation light source 2 such that the integrated amount of the energy given to the photosensitive substance 300 by the light (excitation light) of the specific wavelength bandwidth falls within the range equal to or less than the first integrated energy amount 61 (see FIG. 10), when generating the fluorescence distribution image 41 before the treatment or after the treatment. Note that the first integrated energy amount 61 is an integrated value of the irradiation intensity of the light (excitation light) of the specific wavelength bandwidth emitted to the photosensitive substance 300 and the irradiation time of the light (excitation light) of the specific wavelength bandwidth, when generating the fluorescence distribution image 41 before the treatment or after the treatment.

The first integrated energy amount 61 is determined within a range equal to or less than the predetermined integrated amount of the energy that does not cause necroses of the tumor cells 201 (does not start causing necroses of the tumor cells 201). For example, in a case where the predetermined integrated amount of energy that does not cause necroses of the tumor cells 201 is 0.1 J/cm², the first integrated energy amount 61 is determined such that the integrated amount of energy falls within a range equal to or less than 0.1 J/cm².

As shown in FIG. 10, before the treatment or after the treatment by the photodynamic therapy that causes necroses of the tumor cells 201 by the active oxygen 400 generated based on the chemical reaction, the excitation light source 2 emits the light of the specific wavelength bandwidth such that the first integrated energy amount 61 that is an integrated amount of the energy given by the light of the specific wavelength bandwidth to the photosensitive substance 300 when the image collection unit 4 generates the fluorescence distribution image 41 becomes less than the second integrated energy amount 62 (does not reach the second integrated energy amount 62) that is the integrated amount of the energy given by the light of the specific wavelength bandwidth to the photosensitive substance 300 at the time of the treatment by the photodynamic therapy. The second integrated energy amount 62 is an integrated value of the irradiation strength of the light (therapeutic light) of the specific wavelength bandwidth emitted to the photosensitive substance 300 and the irradiation time of the light (therapeutic light) of the specific wavelength bandwidth at the time of the treatment by the photodynamic therapy that causes necroses of the tumor cells 201 by the active oxygen 400 generated based on the photochemical reaction.

Here, as described above, the photosensitive substance 300 does not emit fluorescence after being changed by the photochemical reaction. Thus, as shown in FIG. 10, as the treatment for causing necroses of the tumor cells 201 based on the irradiation of the light of the specific wavelength bandwidth to the photosensitive substance 300 progresses, the fluorescence intensity (photosensitive substance 300 that emits fluorescence) decreases.

The first integrated energy amount 61 given to the photosensitive substance 300 by the light of the specific wavelength bandwidth emitted from the excitation light source 2 when generating the fluorescence distribution image 41 is an integrated amount of energy within the range with less decrease (attenuation of fluorescence intensity) in the fluorescence intensity per hour than during the treatment. That is, the first integrated energy amount 61 given to the photosensitive substance 300 by the light of the specific wavelength bandwidth emitted from the excitation light source 2 when generating the fluorescence distribution image 41 is an integrated amount of the energy within the range in which the reduction rate of the fluorescence intensity detected by the fluorescence detection unit 13 in accordance with the increase in the integrated amount of energy given to the photosensitive substance 300 becomes less than the reduction rate of the fluorescence intensity during the treatment detected by the fluorescence detection unit 13 in accordance with the increase in the integrated amount of the energy during the treatment given to the photosensitive substance 300 by the light of the specific wavelength bandwidth, as shown in FIG. 10.

Note that although FIG. 10 shows the decreasing tendency in the fluorescence intensity linearly, the decrease in the fluorescence intensity is not limited to a linear decrease, and the fluorescence intensity may decrease while repeating increase and the decrease. Note that the fluorescence intensity in the first integrated energy amount 61 is not limited to being constant and may increase or decrease.

Further, the magnitude of the first integrated energy amount 61 is equal to or greater than a magnitude capable of exciting the photosensitive substance 300 to generate fluorescence and less than a magnitude causing necroses of the tumor cells 201 by the active oxygen 400 generated by the photochemical reaction of the photosensitive substance 300. That is, the magnitude of the first integrated energy amount 61 is a magnitude that the fluorescence detection unit 13 can detect the fluorescence emitted from the photosensitive substance 300 and that the photosensitive substance 300 does not generate active oxygen 400 or generates only a slight amount of active oxygen 400. Thus, the active oxygen 400 does not cause necroses of the tumor cells 201, and even if necroses of the tumor cells 201 have occurred, which is very limited.

Further, the control unit 7 is configured to control the irradiation of the light of the specific wavelength bandwidth by the excitation light source 2 to emit the light of the specific wavelength bandwidth with a predetermined pulse width based on the first integrated energy amount 61 when generating the fluorescence distribution image 41 before the treatment or after the treatment. The control unit 7 controls the irradiation of the excitation light by the excitation light source 2 so as to emit the light (excitation light) of the specific wavelength bandwidth with a predetermined pulse width every time the irradiation is performed by the user. Further, the fluorescence detection unit 13 is configured to detect the fluorescence in synchronization with the predetermined pulse at which the excitation light is emitted. Therefore, the minimum value of the predetermined pulse width is changed based on the value of the rate of the image processing in the fluorescence detection unit 13.

Then, the irradiation intensity of the light of the specific wavelength bandwidth emitted when generating the fluorescence distribution image 41 before the treatment or after the treatment is equal to or greater than the irradiation intensity of the light emitted at the time of the treatment. Specifically, in a case where the irradiation intensity of the light (therapeutic light) of the specific wavelength emitted at the time of the treatment is 150 mW/cm², the irradiation intensity of the light (excitation light) of the specific wavelength emitted when generating the fluorescence distribution image 41 before the treatment or after the treatment becomes 150 mW/cm² or more. For example, in the treatment support device 100, in a case where the irradiation intensity of the light of the specific wavelength emitted at the time of the treatment is 150 mW/cm², as shown in FIG. 11, the irradiation intensity of the excitation light emitted when generating the fluorescence distribution image 41 is set to 150 mW/cm². Further, the treatment support device 100 can set, by changing the setting by the user, the irradiation intensity of the excitation light emitted when generating the fluorescence distribution image 41 to be greater than the irradiation intensity of the light emitted at the time of the treatment.

Further, the control unit 7 is configured to change, based on the first integrated energy amount 61, the settings of the irradiation time and the irradiation intensity of the excitation light source 2 such that the irradiation intensity of the light (excitation light) of the specific wavelength bandwidth emitted from the excitation light source 2 becomes maximum based on the number of times of imaging set (inputted) by the user.

That is, as the number of times of imaging increases, the setting of the control of the irradiation of the light (excitation light) of a specific wavelength bandwidth by the excitation light source 2 is changed so that the irradiation time t per pulse (see FIG. 11) is shortened. In a case where the irradiation time t (pulse width) per pulse reaches the rate of the image processing in the fluorescence detection unit 13, the settings of the control of the irradiation of the light (excitation light) of the specific wavelength bandwidth by the excitation light source 2 is changed to reduce the irradiation intensity.

Further, the control unit 7 can set the irradiation intensity, the irradiation time, and the number of irradiations, respectively, within a range not exceeding the first integrated energy amount 61, by the operation of the user. That is, the treatment support device 100 is configured such that the settings of the irradiation intensity, the irradiation time, and the number of irradiations of the excitation light can be switchable between the settings by the operation by the user (manual setting) and the settings by the control unit 7 (automatic setting).

Further, in this embodiment, the control unit 7 is configured to perform control to restrict the irradiation intensity, the irradiation time, and the number of irradiations of the light of the specific wavelength bandwidth by the excitation light source 2, based on the first integrated energy amount 61. Specifically, the control unit 7 is configured to automatically limit (stop) the irradiation of the light (excitation light) of the specific wavelength bandwidth by the excitation light source 2, in a case where the integrated amount of energy of the excitation light emitted during the observation of the fluorescence distribution before the treatment or after the treatment is likely to exceed the integrated amount (observation upper limit amount) of the energy determined based on the first integrated energy amount 61, in order to prevent the treatment from proceeding during the observation (when generating the fluorescence distribution image 41 before the treatment or after the treatment) of the fluorescence distribution before the treatment or after the treatment. Note that the integrated amount (observation upper limit) of the energy determined based on the first integrated energy amount 61 may be the same as that of the first integrated energy amount 61, or may be less than the wavelength of the first integrated energy amount 61 as long as it is an integrated amount of energy capable of exciting the photosensitive substance 300.

Specifically, in the case where when the irradiation of the excitation light is performed by a determined pulse width every time the user operates, the integrated amount (observation upper limit) of the energy determined based on the first integrated energy amount 61 has reached the upper limit of the number of irradiation that can be irradiated, the control unit 7 is configured to perform control to limit the irradiation of the excitation light so as not to emit the excitation light even if the user performs the operation.

Further, in a case where the irradiation time of the excitation light is long and the integrated amount of the energy given to the photosensitive substance 300 by the excitation light is likely to exceed the integrated amount (observation upper limit) of the energy determined based on the first integrated energy amount 61 when the excitation light is being continuously emitted, the control unit 7 is configured to control the irradiation of the excitation light by stopping (turning off the excitation light 2) the irradiation of the excitation light.

Further, in a case where the irradiation intensity of the excitation light is large and the integrated amount of the energy given to the photosensitive substance 300 by the irradiation of the excitation light exceeds the integrated amount (observation upper limit) of the energy determined based on the first integrated energy amount 61, the control unit 7 is configured to perform control to limit (reduce the irradiation intensity of the excitation light) the irradiation intensity of the excitation light.

Note that the limitation of the irradiation of the excitation light by the control unit 7 may be cancelled in accordance with the operation by the user as needed. Further, as described above, the control of the irradiation of the excitation light by the control unit 7 is configured to perform not only to the excitation light source 2 but also to the excitation light source 2 via the control unit 7 or the like.

### Effects of this Embodiment

In this embodiment, the following effects can be obtained.

According to this embodiment, in the photodynamic therapy, irradiation of the light of the specific wavelength bandwidth having energy capable of generating fluorescence from the photosensitive substance 300 without causing necroses of the tumor cells 201 to the photosensitive substance 300 administered to the body of the patient 200 (subject) is performed by the excitation light source 2 (light source). Further, the information (fluorescence distribution image 41) on the distribution state of the fluorescence generated from the photosensitive substance 300 is outputted from the image collection unit 4 (distribution information output unit) based on the fluorescence generated from the photosensitive substance 300. With this, the information (fluorescence distribution image 41) on the distribution state of the fluorescence generated from the photosensitive substance 300 is outputted without causing necroses of the tumor cells 201 (without advancing the treatment) before the treatment or after the treatment. Therefore, it is possible to confirm the distribution state of the photosensitive substance 300 accumulated in the tumor cells 201 before the treatment or after the treatment by photodynamic therapy.

Further, in the treatment support device 100 according to this embodiment, the following further effects can be obtained by the following configuration.

Further, in the treatment support device 100 according to this embodiment, the fluorescence detection unit 13 is configured to detect light having a wavelength longer than the wavelength of the light of the specific wavelength bandwidth emitted from the excitation light source 2 without detecting the light of the specific wavelength bandwidth emitted from the excitation light source 2 (light source) and light having a wavelength shorter than the wavelength of the light of the specific wavelength bandwidth. With this configuration, at the time of the detection by the fluorescence detection unit 13, the light of the specific wavelength bandwidth emitted from the excitation light source 2 and the light having a wavelength shorter than the wavelength of the light of the specific wavelength bandwidth are excluded, and therefore, the detection of the fluorescence caused by the photosensitive substance 300 can be accurately performed. Consequently, it is possible to accurately confirm the distribution state of the photosensitive substance 300 accumulated in the tumor cells 201.

Further, in the treatment support device 100 according to this embodiment, the fluorescence detection unit 13 is configured to detect the light of the fluorescence of the wavelength bandwidth emitted from talaporfin sodium that is the photosensitive substance 300, without detecting the light of the specific wavelength bandwidth emitted from the excitation light source 2 (light source) and light having a wavelength shorter than the wavelength of the light of the specific wavelength bandwidth. With this configuration, at the time of the detection by the fluorescence detection unit 13, it is possible to detect the light of a specific wavelength bandwidth emitted from the photosensitive substance 300 (talaporfin sodium) in a state in which the light of the specific wavelength bandwidth emitted from the excitation light source 2 and the light having a wavelength shorter than the light of the specific wavelength bandwidth are excluded. Therefore, it is possible to detect the fluorescence due to the photosensitive substance 300 (talaporfin sodium) with higher accuracy. Consequently, it is possible to more accurately confirm the distribution state of the photosensitive substance 300 accumulated in the tumor cells 201.

Further, in the treatment support device 100 according to this embodiment, before the treatment or after the treatment by the photodynamic therapy for causing necroses of the tumor cells 201 by the active oxygen 400 generated based on the photochemical reaction, when the image collection unit 4 (distribution information output unit) generates the fluorescence distribution image 41, the excitation light source 2 (light source) performs the irradiation of the light of the specific wavelength bandwidth such that when the image collection unit 4 (distribution information output unit) generates the fluorescence distribution image 41, the first integrated energy amount 61 that is an integrated amount of the energy given by the light of the specific tumor cell to the photosensitive substance 300 becomes less than the second integrated energy amount 62 that is an integrated amount of the energy given by the light of the specific wavelength bandwidth to the photosensitive substance 300 at the time of the treatment by the photodynamic therapy. With this, before the treatment or after the treatment, when generating the fluorescence distribution image 41, the integrated amount (first integrated energy amount 61) of the energy given by the light of the specific wavelength bandwidth to the photosensitive substance 300 does not reach the integrated amount (second integrated energy amount 62) of the energy given by the light of the specific wavelength bandwidth to the photosensitive substance 300 at the time of the treatment. Consequently, when generating the fluorescence distribution image 41 before the treatment, it is possible to prevent the treatment from being unintentionally started, and when generating the fluorescence distribution image 41 after the treatment, it is possible to prevent the treatment from being unintentionally restarted.

Further, in the treatment support device 100 according to this embodiment, the magnitude of the first integrated energy amount 61 is equal to or greater than the magnitude capable of exciting the photosensitive substance 300 to generate the fluorescence and is less than the magnitude in which the active oxygen 400 generated by the photochemical reaction of the photosensitive substance 300 causes necroses of the tumor cells 201. Accordingly, it is possible to detect the fluorescence generated from the photosensitive substance 300 while suppressing the progress of necroses of the tumor cells 201.

Further, in the treatment support device 100 according to this embodiment, when generating the fluorescence distribution image 41, the first integrated energy amount 61 given by the light of the specific wavelength bandwidth emitted from the excitation light source 2 (light source) is an integrated amount of the energy within the range in which the reduction rate of the fluorescence intensity detected by the fluorescence detection unit 13 in accordance with the increase in the integrated amount of the energy given to the photosensitive substance 300 becomes less than the reduction rate of the fluorescence intensity during the treatment detected by the fluorescence detection unit 13 in accordance with the increase of the integrated amount of the energy during the treatment given to the photosensitive substance 300 by the light of the specific wavelength bandwidth. Thus, when generating the fluorescence distribution image 41, the reduction rate of the fluorescence intensity in accordance with the increase in the integrated amount of the energy becomes less than the reduction rate of the fluorescence intensity during the treatment in accordance with the increase in the integrated amount of the energy during the treatment. Consequently, it is possible to confirm the distribution 40 of the fluorescence generated from the photosensitive substance 300 from the progress of the treatment to the reduction of the fluorescence intensity.

Further, in the treatment support device 100 according to this embodiment, the first integrated energy amount 61 is an integrated value of the irradiation intensity of the light of the specific wavelength bandwidth emitted to the photosensitive substance 300 and the irradiation time of the light of the specific wavelength bandwidth when generating the fluorescence distribution image 41 before the treatment or after the treatment. Further, the second integrated energy amount 62 is an integrated value of the irradiation intensity of the light of the specific wavelength bandwidth emitted to the photosensitive substance 300 and the irradiation time of the light of the specific wavelength bandwidth, at the time of the treatment. The control unit 7 is configured to control the irradiation of the light of the specific treatment by the excitation light source 2 (light source) such that when generating the fluorescence distribution image 41 before the treatment or after the treatment, the integrated value of the energy given to the photosensitive substance 300 by the light of the specific wavelength bandwidth falls within the range equal to or less than the first integrated energy amount 61. With this, the first integrated energy amount 61 is based on the integrated value of the irradiation intensity and the irradiation time, and therefore, it is easily possible to control such that the integrated amount of the energy given to the photosensitive substance 300 by the light (excitation light) of the specific wavelength bandwidth falls within the range equal to or less than the first integrated energy amount 61, when generating the fluorescence distribution image 41 before the treatment or the after the treatment. Consequently, the control unit 7 can more easily control the irradiation of the light (excitation light) of the specific wavelength bandwidth such that the first integrated energy amount 61 does not exceed the second integrated energy amount 62 when generating the fluorescence distribution image 41 before the treatment or after the treatment by the photodynamic therapy.

Further, in the treatment support device 100 according to this embodiment, the control unit 7 is configured to perform control to limit the irradiation intensity, the irradiation time, and the number of irradiations of the light of the specific wavelength bandwidth by the excitation light source 2 (light source), based on the first integrated energy amount 61. With this, when generating the fluorescence distribution image 41 before the treatment or after the treatment, it is possible to prevent that the integrated amount of the energy given to the photosensitive substance 300 by the light (excitation light) of the specific wavelength bandwidth exceeds the first integrated energy amount 61 by the increase in the irradiation intensity, the irradiation time, and the number of irradiations of the light (excitation light) of the specific wavelength bandwidth.

Further, in the treatment support device 100 according to this embodiment, the control unit 7 is configured to control the irradiation of the light of the specific wavelength bandwidth by the excitation light source 2 (light source) to emit the light of the specific wavelength bandwidth with a predetermined pulse width based on the first integrated energy amount 61, when generating the fluorescence distribution image 41 before the treatment or after the treatment. With this, the light (excitation light) of the specific wavelength bandwidth is emitted with the pulse width based on the first integrated energy amount 61, and therefore, it becomes possible to shorten the irradiation time of the light (excitation light) of the specific wavelength bandwidth emitted from the excitation light source 2 when generating the fluorescence distribution image 41, before the treatment or after the treatment. Consequently, it is possible to increase the irradiation intensity of the excitation light within the range not exceeding the first integrated energy amount 61.

Further, in the treatment support device 100 of this embodiment, the irradiation intensity of the light of the specific wavelength bandwidth emitted when generating the fluorescence distribution image 41 is equal to or greater than the irradiation intensity of the light emitted at the time of the treatment, before the treatment or after the treatment. With this, before the treatment or after the treatment by photodynamic therapy, when generating the fluorescence distribution image 41, the light (excitation light) of the specific wavelength bandwidth is emitted at the irradiation intensity equal to or greater than the irradiation intensity of the light emitted at the time of the treatment. Therefore, it is possible to confirm the distribution state of the photosensitive substance 300 to the depth position equal to or greater than the depth position at which the light (therapeutic light) emitted at the time of the treatment reaches.

Further, in the treatment support device 100 according to this embodiment, the image collection unit 4 (distribution information output unit) is configured to generate the visible light image 42 based on the visible light detected by the visible light detection unit 14. Further, the PC 5 is configured to generate the composite image 43 in which the fluorescence distribution image 41 and the visible light image 42 are superimposed. Further, the control unit 7 is configured to perform control to display the composite image 43 on the display unit 9. As a result, the composite image 43 in which the fluorescence distribution image 41 and the visible light image 42 are superimposed is displayed on the display unit 9, and therefore, it is possible to easily compare the distribution 40 (fluorescence distribution image 41) of the fluorescence and the visible light image 42.

### Modified Embodiments

It should be understood that the embodiments disclosed here are examples in all respects and are not restrictive. The scope of the present invention is shown by claims rather than the descriptions of the embodiments described above and includes all changes (modifications) within the meaning and range equivalent to the claims.

For example, in the above-described embodiment, an example is shown in which the treatment support device 100 (photodynamic therapy treatment support device) is provided with the display unit 9 for displaying the composite image 43, but the present invention is not limited thereto. In the present invention, the photodynamic therapy treatment support device may be configured not to include a display unit but to output and display the composite image or the like to a monitor or the like outside the device.

Further, in the above-described embodiment, an example is shown in which, before the treatment or after the treatment, the irradiation intensity of the excitation light emitted when generating the fluorescence distribution image 41 is equal to or greater than the irradiation intensity of the light emitted at the time of the treatment, and the excitation light source 2 is configured to perform control to emit the light of the specific wavelength bandwidth, but the present invention is not limited thereto. In the present invention, the photodynamic therapy treatment support device may be configured to continuously emit the excitation light, before the treatment or after treatment or after treatment, by setting the irradiation intensity of the excitation light to be emitted when generating the fluorescence distribution to be less than the irradiation intensity of the light to be emitted at the time of the treatment. For example, as shown in FIG. 12, in a case where the irradiation intensity of the light having a specific wavelength irradiated at the time of the treatment is 150 mW/cm², it may be configured to continuously emit the excitation light by setting the irradiation intensity of the excitation light emitted when generating the fluorescence distribution image to 50 mW/cm².

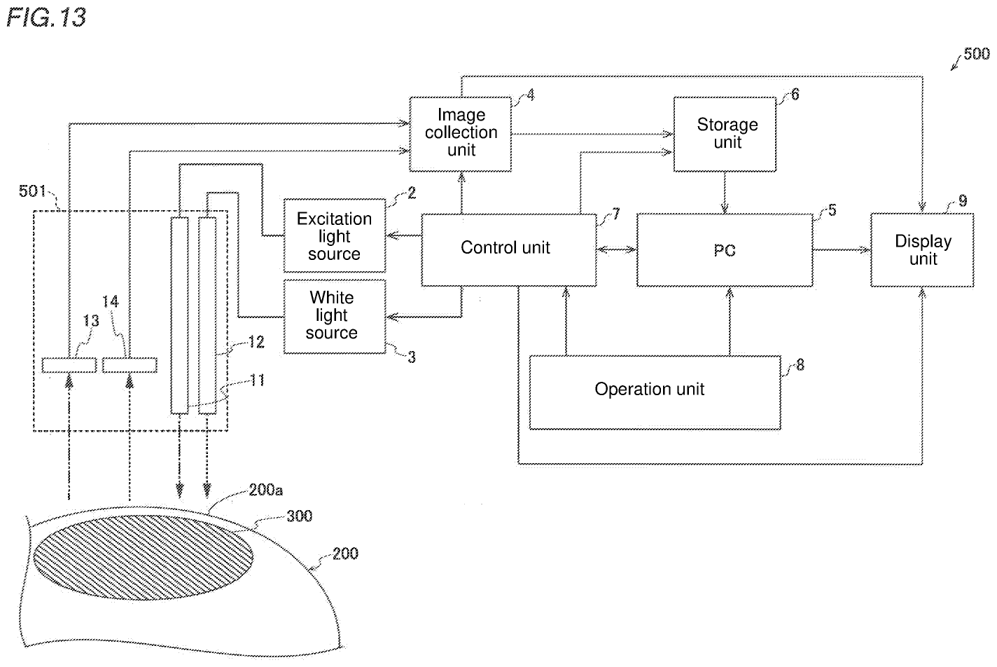

Further, in the above-described embodiment, an example is shown in which the endoscope 1 inserted into the body of the patient 200 performs the irradiation of the light (excitation light and therapeutic light) of the specific wavelength bandwidth and the detection of the fluorescence emitted from the photosensitive substance 300, but the present invention is not limited thereto. In the present invention, it may be configured such that like the photodynamic therapy treatment support device 500 according to a first modification shown in FIG. 13, the light (excitation light and therapeutic light) of the specific wavelength bandwidth is emitted to the affected area 200a exposed by thoracotomy, laparotomy, craniotomy, or the like from the outside of the patient 200 by the irradiation observation unit 501 arranged outside the patient 200 and detect the fluorescence light emitted from the photosensitive substance 300 from the outside of the patient 200. Further, the photodynamic therapy treatment support device may be provided with both the endoscope 1 according to the above-described embodiment and the irradiation observation unit 501 according to the first modification. Further, the photodynamic therapy treatment support device may be configured such that each of the endoscope 1 and the irradiation observation unit 501 are detachable and can be replaced to each other depending on the treatment.

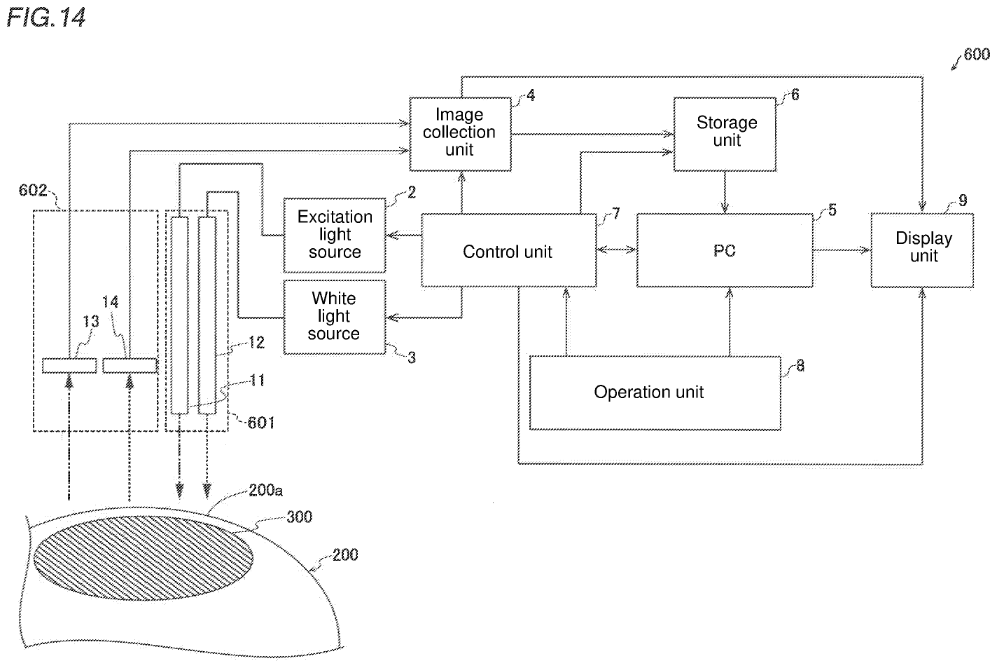

Further, in the above-described embodiment, an example is shown in which the endoscope 1 inserted into the body of the patient 200 performs the irradiation of the specific wavelength bandwidth of the light (excitation light and therapeutic light) and the detection of the fluorescence emitted from the photosensitive substance 300, but the present invention is not limited thereto. In the present invention, the portion that emits the light (excitation light and therapeutic light) of the specific wavelength bandwidth and the portion that detects the fluorescence emitted from the photosensitive substance 300 may be separately provided. For example, like the treatment support device 600 shown in FIG. 14, it may be provided with an irradiation unit 601 including a light guide 11 for guiding the light of the specific wavelength bandwidth emitted from the excitation light source 2, a light guide 12 for guiding the white light emitted from the white light source 3, and an observation unit 602 including the fluorescence detection unit 13 and a visible light detection unit 14.

Further, in the above-described embodiment, an example is shown in which the fluorescence detection unit 13 is configured to detect the light having a wavelength longer than the wavelength of the light of the specific wavelength bandwidth emitted from the excitation light source (light source), without detecting the light of the specific wavelength bandwidth emitted from the excitation light source 2 (light source) and light having a wavelength shorter than the wavelength of the light of the specific wavelength bandwidth emitted from the excitation light source 2 (light source), but the present invention is not limited thereto. In the present invention, the fluorescence detection unit may be configured to detect light having a wavelength shorter than the wavelength of the light of the specific light source and light having a wavelength longer than the wavelength of the light of the specific light source, without detecting the light of the specific wavelength bandwidth irradiated by the light source.

Further, in the above-described embodiment, an example is shown in which the photosensitive substance 300 is talaporfin sodium, but the present invention is not limited thereto. In the present invention, the photosensitive substance may not be talaporfin sodium. The photosensitive substance may be, for example, a chlorine-based medical agent having a chlorine skeleton.

Further, in the above-described embodiment, an example is shown in which the control unit 7 controls the irradiation of the light of the specific wavelength bandwidth by the excitation light source 2 (light source) such that, when generating the fluorescence distribution image 41 before the treatment or after the treatment, the integrated amount of the energy given to the photosensitive substance 300 by the light of the specific wavelength bandwidth falls within the range equal to or less than the first integrated energy amount 61, but the present invention is not limited thereto. In the present invention, the control unit may be configured to perform control to notify the user that when the integrated amount of the energy given to the photosensitive substance by the light of the specific wavelength bandwidth has reached a preset threshold, the integrated amount of the energy given to the photosensitive substance by the light of the specific wavelength bandwidth by the display on the display unit or sound is likely to exceed the first integrated energy amount. And the user may perform the control (stopping the irradiation) of the light (excitation light) of the specific wavelength bandwidth based on the notification.

Further, in the above-described embodiment, an example is shown in which the treatment support device 100 (photodynamic therapy treatment support device) is configured to perform treatment in addition to the support of the treatment, but the present invention is not limited thereto. In the present invention, the photodynamic therapy treatment support device may be configured to only support the treatment by photoimmunotherapy (only generate the fluorescence distribution image or only output the information on the distribution state of the fluorescence).

Further, in the above-described embodiment, an example is shown in which the image collection unit 4 (distribution information output unit) is configured to generate, based on the fluorescence generated from the photosensitive substance 300, the fluorescence distribution image 41, which is an image representing the distribution state of the fluorescence generated from the photosensitive substance 300, but the present invention is not limited thereto. In the present invention, the distribution information output unit may be configured to output the value (fluorescence signal strength) of the detected fluorescence signal as a numerical value, or may be configured to output the mean value of the value of the detected fluorescence signal (fluorescence signal strength).

In the above-described embodiment, an example is shown in which the treatment support device 100 (photodynamic therapy treatment support device) is separately provided with the fluorescence detection unit 13 and the visible light detection unit 14, but the present invention is not limited thereto. In the present invention, the photodynamic therapy treatment support device may be configured to detect the fluorescence emitted from the photosensitive substance by the irradiation of the light of the specific wavelength bandwidth and the visible light with a common detection unit (image sensor).

### ASPECTS

It will be understood by those skilled in the art that the above-described exemplary embodiments are concrete examples of the following aspects.

### (Item 1)

A photodynamic therapy treatment support device for use in photodynamic therapy for causing necroses of tumor cells by active oxygen generated by a photochemical reaction caused by irradiating a photosensitive substance with light of a specific wavelength bandwidth, the photodynamic therapy treatment support device comprising:

a light source configured to irradiate the photosensitive substance administered to an object with the light of the specific wavelength bandwidth, the light having energy capable of generating fluorescence from the photosensitive substance without advancing necrosis of the tumor cell; and

a distribution information output unit configured to output information on a distribution state of the fluorescence generated from the photosensitive substance, based on the fluorescence generated from the photosensitive substance.

### (Item 2)

The photodynamic therapy treatment support device as recited in the above-described Item 1, further comprising:

a fluorescence detection unit configured to detect the fluorescence emitted from the photosensitive substance by being irradiated with the light of the specific wavelength bandwidth,

wherein the fluorescence detection unit is configured to detect light having a wavelength longer than a wavelength of the light of the specific wavelength bandwidth emitted from the light source, without detecting the light of the specific wavelength bandwidth emitted from the light source and light having a wavelength shorter than the wavelength of the light of the specific wavelength bandwidth.

### (Item 3)

The photodynamic therapy treatment support device as recited in the above-described Item 2,

Wherein the fluorescence detection unit is configured to detect the light of fluorescence of a wavelength bandwidth emitted from the photosensitive substance, without detecting the light of the wavelength bandwidth emitted from the light source and light having a wavelength shorter than the wavelength of the light of the specific wavelength bandwidth.

### (Item 4)

The photodynamic therapy treatment support device as recited in the above-described Item 3,

wherein the photosensitive substance is talaporfin sodium.

### (Item 5)

The photodynamic therapy treatment support device as recited in the above-described Items 2 to 4,

wherein the distribution information output unit is configured to generate a fluorescence distribution image based on the fluorescence generated from the photosensitive substance, the fluorescence distribution image being an image representing a distribution state of the fluorescence generated from the photosensitive substance, and

wherein the light source is configured to emit the light of the specific wavelength bandwidth such that a first integrated energy amount becomes less than a second integrated energy amount, the first integrated energy amount being an integrated amount of energy given by the light of the specific wavelength bandwidth to the photosensitive substance when generating the fluorescence distribution image before the treatment or after treatment by the photodynamic therapy for causing necroses of the tumor cell by active oxygen generated by the photochemical reaction, the second integrated energy amount being an integrated amount of energy given by light of the specific wavelength bandwidth to the photosensitive substance at the time of the treatment by the photodynamic therapy.

### (Item 6)

The photodynamic therapy treatment support device as recited in the above-described Item 5,

wherein magnitude of the first integrated energy amount is equal to or greater than magnitude capable of exciting the photosensitive substance to generate the fluorescence and less than magnitude causing necrosis of the tumor cell by the active oxygen generated by the photochemical reaction of the photosensitive substance is less than magnitude.

### (Item 7)

The photodynamic therapy treatment support device as recited in the above-described Item 5 or 6,

wherein the first integrated energy amount given to the photosensitive substance by the light of the specific wavelength bandwidth emitted from the light source when generating the fluorescence distribution image is an integrated amount of energy within a range in which a reduction range of fluorescence intensity detected by the fluorescence detection unit in accordance with an increase in an integrated amount of energy applied to the photosensitive substance becomes less than a reduction rate of fluorescence intensity during the treatment detected by the fluorescence detection unit in accordance with an increase in an integrated amount of energy applied to the photosensitive substance by the light of the specific wavelength bandwidth during the treatment.

### (Item 8)

The photodynamic therapy treatment support device as recited in any one of the above-described Items 5 to 7, further comprising:

a control unit configured to control irradiation of the light of the specific wavelength bandwidth by the light source;

wherein the first integrated energy amount is an integrated value of irradiation intensity of the light of the specific wavelength bandwidth and an irradiation time of the light of the specific wavelength bandwidth when generating the fluorescence distribution image before and after the treatment,

wherein the second integrated energy amount is an integrated value of irradiation intensity of the light of the specific wavelength bandwidth emitted to the photosensitive substance and an irradiation time of the light of the specific wavelength bandwidth at the time of the treatment, and

wherein the control unit is configured to control irradiation of the light of the specific wavelength bandwidth by the light source such that an integrated amount of the energy given to the photosensitive substance by the light of the specific wavelength bandwidth when generating the fluorescence distribution image before and after the treatment becomes equal to or less than the first integrated energy amount.

### (Item 9)

The photodynamic therapy treatment support device as recited in the above-described Item 8,

wherein the control unit is configured to perform control to limit the irradiation intensity, the irradiation time, and the number of irradiations of the light of the specific wavelength bandwidth by the light source, based on the first integrated energy amount.

### (Item 10)

The photodynamic therapy treatment support device as recited in the above-described Item 8 or 9,

wherein the control unit is configured to control the irradiation of the light of the specific wavelength bandwidth by the light source such that the light of the specific wavelength bandwidth is emitted with a predetermined pulse width based on the first integrated energy amount when generating the fluorescence distribution image before the treatment or after the treatment.

### (Item 11)

The photodynamic therapy treatment support device as recited in the above-described Item 10,

wherein the irradiation intensity of the light of the specific wavelength bandwidth emitted when generating the fluorescence distribution image before and after the treatment is equal to or greater than irradiation intensity of the light emitted at the time of the treatment.

### (Item 12)

The photodynamic therapy treatment support device as recited in any one of the above-described Items 8 to 11, further comprising:

a visible light detection unit configured to detect visible light;

an image composition unit configured to generate a composite image in which a plurality of images generated by the distribution information output unit is superimposed; and

a display unit configured to display the composite image,

wherein the distribution information output unit is configured to generate a visible image based on the visible light detected by the visible image detection unit,

wherein the image composite unit is configured to generate the composite image in which the fluorescence distribution image and the visible light image are superimposed, and

wherein the control unit is configured to perform control to display at least the composite image on the display unit.

1. A photodynamic therapy treatment support device for use in photodynamic therapy for causing necroses of tumor cells by active oxygen generated based on a photochemical reaction caused by irradiating a photosensitive substance with light of a specific wavelength bandwidth, the photodynamic therapy treatment support device comprising:
a light source configured to irradiate the photosensitive substance administered to a body of a subject with the light of the specific wavelength bandwidth, the light having energy capable of generating fluorescence from the photosensitive substance without causing necroses of the tumor cells; and
a distribution information output unit configured to output information on a distribution state of the fluorescence generated from the photosensitive substance, based on the fluorescence generated from the photosensitive substance.

2. The photodynamic therapy treatment support device as recited in claim 1, further comprising:
a fluorescence detection unit configured to detect the fluorescence emitted from the photosensitive substance by being irradiated with the light of the specific wavelength bandwidth,
wherein the fluorescence detection unit is configured to detect light having a wavelength longer than a wavelength of the light of the specific wavelength bandwidth emitted from the light source, without detecting the light of the specific wavelength bandwidth emitted from the light source and light having a wavelength shorter than the wavelength of the light of the specific wavelength bandwidth.

3. The photodynamic therapy treatment support device as recited in claim 2,
wherein the fluorescence detection unit is configured to detect the light of fluorescence of a wavelength bandwidth emitted from the photosensitive substance, without detecting the light of the wavelength bandwidth emitted from the light source and light having a wavelength shorter than the wavelength of the light of the specific wavelength bandwidth.

4. The photodynamic therapy treatment support device as recited in claim 3,
wherein the photosensitive substance is talaporfin sodium.

5. The photodynamic therapy treatment support device as recited in claim 2,
wherein the distribution information output unit is configured to generate a fluorescence distribution image based on the fluorescence generated from the photosensitive substance, the fluorescence distribution image being an image representing the distribution state of the fluorescence generated from the photosensitive substance, and
wherein the light source is configured to emit the light of the specific wavelength bandwidth such that before treatment or after treatment by the photodynamic therapy for causing necroses of the tumor cells by the active oxygen generated based on the photochemical reaction, a first integrated energy amount becomes less than a second integrated energy amount, the first integrated energy amount being an integrated amount of energy given by the light of the specific wavelength bandwidth to the photosensitive substance when generating the fluorescence distribution image, the second integrated energy amount being an integrated amount of energy given by the light of the specific wavelength bandwidth to the photosensitive substance at the time of the treatment by the photodynamic therapy.

6. The photodynamic therapy treatment support device as recited in claim 5,
wherein magnitude of the first integrated energy amount is equal to or greater than magnitude capable of exciting the photosensitive substance to generate the fluorescence and less than magnitude causing necroses of the tumor cells by the active oxygen generated by the photochemical reaction of the photosensitive substance.

7. The photodynamic therapy treatment support device as recited in claim 5,
wherein the first integrated energy amount given to the photosensitive substance by the light of the specific wavelength bandwidth emitted from the light source when generating the fluorescence distribution image is an integrated amount of energy within a range in which a reduction range of fluorescence intensity detected by the fluorescence detection unit in accordance with an increase in an integrated amount of energy applied to the photosensitive substance becomes less than a reduction rate of fluorescence intensity during the treatment detected by the fluorescence detection unit in accordance with an increase in an integrated amount of energy applied to the photosensitive substance during the treatment by the light of the specific wavelength bandwidth.

8. The photodynamic therapy treatment support device as recited in claim 5, further comprising:
a control unit configured to control irradiation of the light of the specific wavelength bandwidth by the light source;
wherein the first integrated energy amount is an integrated value of irradiation intensity of the light of the specific wavelength bandwidth and an irradiation time of the light of the specific wavelength bandwidth, the light of the specific wavelength bandwidth being emitted to the photosensitive substance when generating the fluorescence distribution image before the treatment or after the treatment,
wherein the second integrated energy amount is an integrated value of irradiation intensity of the light of the specific wavelength bandwidth and an irradiation time of the light of the specific wavelength bandwidth, the light of the specific wavelength bandwidth being emitted to the photosensitive substance at the time of the treatment, and
wherein the control unit is configured to control irradiation of the light of the specific wavelength bandwidth by the light source such that when generating the fluorescence distribution image before the treatment or after the treatment, an integrated amount of the energy given to the photosensitive substance by the light of the specific wavelength bandwidth falls within a range equal to or less than the first integrated energy amount.

9. The photodynamic therapy treatment support device as recited in claim 8,
wherein the control unit is configured to perform control to limit the irradiation intensity, the irradiation time, and the number of irradiations of the light of the specific wavelength bandwidth by the light source, based on the first integrated energy amount.

10. The photodynamic therapy treatment support device as recited in claim 8,
wherein the control unit is configured to control the irradiation of the light of the specific wavelength bandwidth by the light source such that when generating the fluorescence distribution image before the treatment or after the treatment, the light of the specific wavelength bandwidth is emitted with a predetermined pulse width based on the first integrated energy amount.

11. The photodynamic therapy treatment support device as recited in claim 10,
wherein before the treatment or after the treatment, the irradiation intensity of the light of the specific wavelength bandwidth emitted when generating the fluorescence distribution image is equal to or greater than irradiation intensity of the light emitted at the time of the treatment.

12. The photodynamic therapy treatment support device as recited in claim 8, further comprising:
a visible light detection unit configured to detect visible light;
an image composition unit configured to generate a composite image in which a plurality of images generated by the distribution information output unit is superimposed; and
a display unit configured to display the composite image,
wherein the distribution information output unit is configured to generate a visible image based on the visible light detected by the visible image detection unit,
wherein the image composite unit is configured to generate the composite image in which the fluorescence distribution image and the visible light image are superimposed, and
wherein the control unit is configured to perform control to display at least the composite image on the display unit.
